(19) United States
(12) Patent Application Publication
(10) Pub. No.: US 2007/0112251 A1
(43) Pub. Date: May 17, 2007

(54) Apparatuses and methods for delivering one or more deliverables into a body

(75) Inventor: Gary S. Nakhuda, New York, NY (US)

Correspondence Address: RODMAN RODMAN, 7 SOUTH BROADWAY, WHITE PLAINS, NY, 10601, US

(73) Assignee: The Trustees Of Columbia University In The City Of New York, New York, NY (US)

(21) Appl. No.: 11/594,587

(22) Filed: Nov. 8, 2006

Related U.S. Application Data
(60) Provisional application No. 60/734,587, filed on Nov. 8, 2005.
(60) Provisional application No. 60/836,001, filed on Aug. 7, 2006.

(51) Int. Cl. A61B 1/00

(52) U.S. Cl. 600/114; 600/146; 600/104

(57) ABSTRACT

Apparatuses and methods for delivering one or more deliverables into a body are provided. In some embodiments, apparatuses for delivering one or more deliverables into a body are provided, the apparatuses comprising: a sheath; an endoscope having a distal portion and a proximal portion and including a microfiberoptic disposed at the distal portion, said endoscope being capable of being inserted at least partially within the sheath, and capable of guiding insertion of the sheath into a configuration in the body; and a catheter capable of being inserted into the sheath, and capable of delivering one or more deliverables to the location in the body based upon the configuration of the sheath; wherein the inner diameter of the outer sheath is less than the sum of the outer diameter of the microfiberoptic and the outer diameter of the catheter.

### CROSS-REFERENCE TO RELATED APPLICATIONS

This application claims the benefit of U.S. Provisional Patent Application No. 60/734,587, filed on Nov. 8, 2005, and U.S. Provisional Patent Application No. 60/836,001, filed on Aug. 7, 2006, each of which are hereby incorporated by reference herein in their entirety.

### TECHNOLOGY AREA

The disclosed subject matter relates to apparatuses and methods for delivering one or more deliverables into a body.

### BACKGROUND

Assisted reproductive technology (ART) can be utilized to assist women to overcome infertility. In-vitro fertilization (IVF), which is one type of ART, generally involves surgically removing an egg from a female and exposing the egg with sperms in a laboratory dish. If the egg fertilizes and begins cell division, the resulting embryo is transferred into the female's uterus. If implantation of the embryo in the endometrial lining occurs, the embryo will further develop, resulting in a normal pregnancy. Other types of ART include gamete intrafallopian transfer (GIFT), zygote intrafallopian transfer (ZIFT), pronuclear stage tubal transfer (PROST). GIFT involves introducing a mixture of sperms and eggs (gamete) into the fallopian tube where the eggs are fertilized. ZIFT involves fertilizing eggs to form zygotes in vitro and then introducing the zygotes into the fallopian tube. PROST involves fertilizing eggs in vitro and then introducing the fertilized eggs into the fallopian tube before cell division occurs.

An ART procedure may be performed in conjunction with medications that stimulate the ovaries to produce multiple eggs, in order to increase the likelihood of successful fertilization. Also, multiple eggs or embryos/zygotes may be transferred into the female's uterus or fallopian tube to increase the likelihood of successful implantation.

In a GIFT, ZIFT, or PROST procedure, introduction of gamete, zygotes, or fertilized eggs occurs through an incision in the abdomen (laproscopy) which is undesirable. In an IVF procedure, it is difficult to transfer eggs into a female's uterus and implanting the embryo into the endometrial lining of the uterus. To perform this task, an embryo transfer device, typically including a catheter, needs to be inserted to a desired depth in the endometrial cavity. Before the catheter reaches into the endometrial cavity, it must first pass the cervical canal, which can be very difficult because of unusual contours in the canal or dramatic angles between the cervix and the body of the uterus. Difficult and traumatic transfers have been associated with lower IVF pregnancy rates.

Conventionally, insertion of the embryo transfer device is performed blindly (by “feel”), or under transabdominal ultrasound guidance. While ultrasound guidance is frequently helpful for positioning a catheter in the endometrial cavity, it is of little use in negotiating a passage for the catheter through the cervical canal. Ultrasound is also not helpful in all females, especially females with thicker abdominal walls, where resolution of the ultrasound beam can be limited. Blind or ultrasound-guided insertion of a catheter may also result in the creation of “false passages” that cause reproductive complications. Therefore, direct visualization of the cervical canal and the uterus during the insertion of the embryo transfer device is desirable.

Direct visualization can be enabled by the use of hysteroscopes, which are intrauterine endoscopes that allow visualization of the uterus. However, because of the size of conventional hysteroscopes, dilation of the cervix may be necessary, and the patients may require anesthesia during the procedure. However, dilation of the cervix is highly undesirable at the time of an embryo transfer.

### SUMMARY

Apparatuses and methods for delivering one or more deliverables into a body are provided. In some embodiments, apparatuses for delivering one or more deliverables into a body are provided, the apparatuses comprising: a sheath; an endoscope having a distal portion and a proximal portion and including a microfiberoptic disposed at the distal portion, said endoscope being capable of being inserted at least partially within the sheath, and capable of guiding insertion of the sheath into a configuration in the body; and a catheter capable of being inserted into the sheath, and capable of delivering one or more deliverables to the location in the body based upon the configuration of the sheath; wherein the inner diameter of the outer sheath is less than the sum of the outer diameter of the microfiberoptic and the outer diameter of the catheter.

In some embodiments, methods for delivering one or more deliverables into a body are provided, the methods comprising: inserting an endoscope inside a sheath; inserting the endoscope and the sheath into the body; removing the endoscope from the sheath while leaving the sheath in the body; inserting a catheter capable of carrying one or more deliverables into the sheath; and delivering the deliverables to the location in the body using the catheter.

In some embodiments, apparatuses for delivering one or more deliverables into a body, the apparatuses comprising: means for inserting an endoscope inside a sheath; means for inserting the endoscope and the sheath into the body; means for removing the endoscope from the sheath while leaving the sheath in the body; means for inserting a catheter capable of carrying one or more deliverables into the sheath; and means for delivering the deliverables to the location in the body using the catheter.

### BRIEF DESCRIPTION OF THE DRAWINGS

FIG. 1 is a side view of a microfiberoptic endoscope of a microfiberoptic transfer catheter of in accordance with some embodiments of the disclosed subject matter.

FIG. 2A is a side view of an outer sheath of a microfiberoptic transfer catheter of in accordance with some embodiments of the disclosed subject matter.

FIG. 2B is a cross sectional view of an outer sheath of a microfiberoptic transfer catheter of in accordance with some embodiments of the disclosed subject matter.

FIG. 3 is a side view of an inner catheter of a microfiberoptic transfer catheter of in accordance with some embodiments of the disclosed subject matter.

FIG. 4 is a side view illustrating a first stage in a deliverable transfer method utilizing a microfiberoptic transfer catheter of in accordance with some embodiments of the disclosed subject matter.

FIG. 5 is a side view illustrating a second stage in deliverable transfer method utilizing a microfiberoptic transfer catheter of in accordance with some embodiments of the disclosed subject matter.

FIG. 6 is a side view illustrating a third stage in a deliverable transfer method utilizing a microfiberoptic transfer catheter of in accordance with some embodiments of the disclosed subject matter.

### DETAILED DESCRIPTION

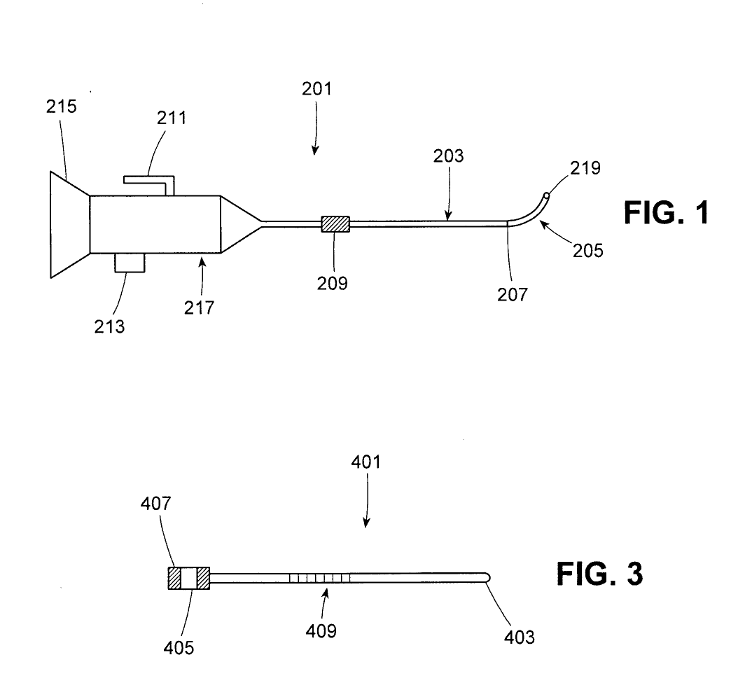

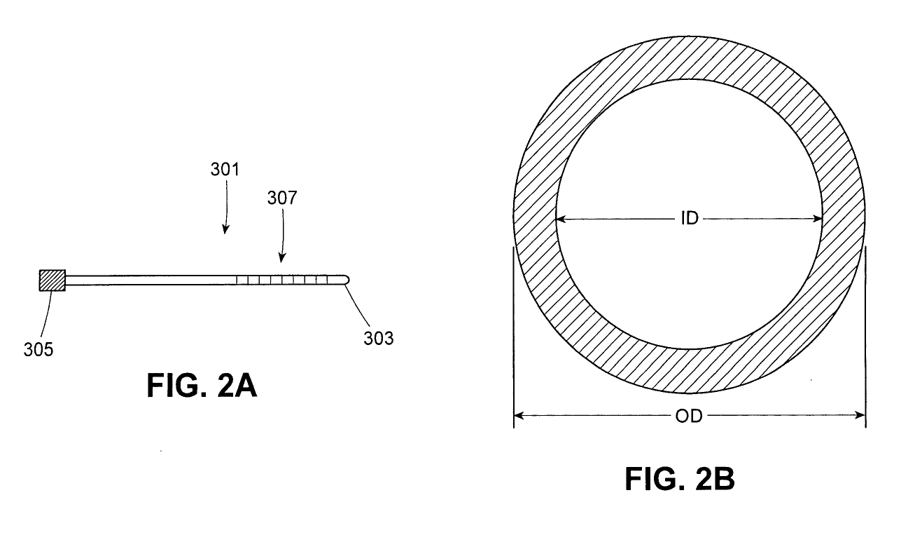

Apparatuses and methods for delivering one or more deliverables into a body are provided. In some embodiments, a microfiberoptic transfer catheter can include at least the following three components: a steerable, flexible microfiberoptic endoscope (see FIG. 1); a flexible outer sheath (see FIGS. 2A and 2B), inside which the endoscope can be fitted; and a flexible inner catheter (see FIG. 3), which can fit inside the outer sheath.

FIG. 1, shows a side view of a microfiberoptic endoscope 201. According to some embodiments, the endoscope 201 can include a microfiberoptic 203, a distal tip 205, a demarcator 207, a connector 209, a steering lever 211, a light source 213, a coupler 215, an eyepiece 217, and lens 219. The catheter can be in any size or dimension as long as it is configured to be inserted into a desired location of the human body.

In some embodiments, the microfiberoptic 203 can be approximately 25 centimeters long. The outer diameter of the microfiberoptic 203 can be any suitable size. For example, the outer diameter of the microfiberoptic 203 can be approximately 1.5 millimeters when utilized to deliver deliverables into the uterus. As another example, the outer diameter of the microfiberoptic 203 can be approximately 0.05 millimeters when utilized to deliver deliverables into the intramural portion of the fallopian tube. Other suitable sizes for desired delivery location will be readily apparent to one of ordinary skill in the art. The microfiberoptic 203 can be flexible for easier and less traumatic insertion.

The distal tip 205 of the microfiberoptic 203 can be of any suitable size. For example, distal tip 205 can be approximately 1-2 centimeters long. Various sizes and dimensions are provided herein only as examples. A lens 219 can be located at the distal end of the distal tip 205. Endoscope 201 can have any suitable angle of view. For example, 0 degrees, 10 degrees, 30 degrees, 70, degrees and the like can be suitable. In some embodiments, the angle of view of endoscope 201 can vary. For example, the angle of view can vary from 0 to 90 degrees, from 20 to 80 degrees, from 30 to 70 degrees, and the like. The angle of view can be determined by the cant of lens 219.

A demarcator 207 on the microfiberoptic 203 can indicate the proximal end of the flexible tip 205. A connector 209 can be located on the microfiberoptic 203. Connector 209 can be any suitable mechanism for connecting two parts. For example, a suitable connector 209 can be a locking mechanism, such as a Luer lock. In some embodiments, the position of the connector 209 is adjustable. Alternatively, the connector 209 can be fixed on the microfiberoptic 203.

An eyepiece 217 can also be located at the proximal portion of the endoscope 201. Eyepiece 217 can include one or more lens element. A user may look through eyepiece 217 directly. A coupler 215 can also be attached to the eyepiece 217 and can be located at the proximal end of the endoscope 201. The coupler 215 can be used to couple optically the eyepiece 217 with a video monitoring device (not shown). The video monitoring device can, for example, include a video camera, which can be further attached to a video monitor. Any suitable video equipment can be used. For example, video equipment that can be used is commercially available and currently in use for other endoscopic applications. For example, the video equipment can be a TRICAM camera head and a Medi Pack terminal from Karl Storz Endoscopy-America, Inc., Culver City, Calif., or a Visera System from Olympus America, Inc., Melville, N.Y. The microfiberoptic 203 can be disinfected and sterilized in gas or liquid solution.

A steering lever 211 and a light source 213 can be attached to the eyepiece 217. The distal tip 205 can be bent by the steering lever 211. Bending of the distal tip 205 can allow easier and less traumatic insertion of the endoscope 201. In some embodiments, the distal tip 205 can be bent because the microfiberoptic 203 includes small flexible wires that are attached to the steering lever 211 on the eyepiece 217 of the endoscope 201. Pulling the lever 211 in one direction pulls the wires (not shown) in that direction, causing distal tip 205 to bend in the same direction. This bending or steering mechanism can be the same as that used in other endoscopes, such as endoscopes used for colonoscopy or other gastrointestinal applications.

Another component of the microfiberoptic embryo transfer catheter is illustrated in FIG. 2A, which is a side view of the outer sheath 301. FIG. 2B is a cross-sectional view of the outer sheath 301. The outer sheath 301 is preferably made of non-toxic polymers safe for medical use, such as polyethylene, polyvinylchloride, polypropylene, polystyrene and/or any other suitable material. Outer sheath 301 can be designed for a single use. The outer sheath 301 can be flexible for easier and less traumatic insertion. In some embodiments, the distal tip 303 can be slightly beveled. A beveled tip allows easier and less traumatic insertion than a tip with a blunt end.

In some embodiments, the distal tip 303 can also be echolucent so that the position of the distal tip 303 of the outer sheath 301 can be confirmed by ultrasound. A threaded locking mechanism 305 can be located at the proximal end of the outer sheath 301 so that the outer sheath 301 can be secured to the microfiberoptic endoscope 201 illustrated in FIG. 1, using the connector 209 located on the microfiberoptic endoscope 201. Locking mechanism 305 can be, for example, a Luer lock. In some embodiments, the outer surface of the outer sheath 301 can be demarcated (shown as 307) with gradations for every centimeter (or any other unit of measure) starting from the distal tip 303. The outer sheath 301 can be designed in different lengths (e.g., 12-18 centimeters) to accommodate variations in pelvic anatomy. In some embodiments, the connector 209 on the microfiberoptic endoscope 201 can be adjusted to accommodate different lengths of the outer sheath 301.

As FIG. 2B illustrates, the outer sheath 301 can have an inner diameter (ID) that is slightly larger than the outer diameter (OD) of microfiberoptic 203. For example, if the outer diameter (OD) of microfiberoptic 203 is about 1.5 millimeters, the inner diameter (ID) of outer sheath 301 can be approximately 1.6 millimeters, so that the microfiberoptic 203 can be fitted within the outer sheath 301. The outer diameter (OD) of the outer sheath 301 can be less than about several millimeters. For example, the outer diameter (OD) of the outer sheath 301 can be about 1.9 millimeters. Although 1.9 millimeters is not meant to be the absolute maximum, a small outer diameter (OD) of the outer sheath 301 can reduce the difficulty and trauma associated with the insertion of the device.

Yet another component of the microfiberoptic embryo transfer catheter is illustrated in FIG. 3, which is a side view of the inner catheter 401. The inner catheter 401 can also be made of nontoxic polymer safe for medical use, such as polyethylene, polyvinylchloride, polypropylene, polystyrene and/or any other suitable material. In some embodiments, one inner catheter can be packaged together with one outer sheath for a single use. The outer diameter of the inner catheter 401 can be of any suitable size capable of fitting inside the inner diameter of the outer sheath 301. For example, if the inner diameter of outer sheath 301 is about 1.6 millimeters, the outer diameter of inner catheter 401 can be approximately 1.5 millimeters, so that it can be fitted inside the outer sheath 301. The inner catheter 401 can also be flexible, so that it can be inserted into the outer sheath 301 when the outer sheath 301 is contorted.

The length of the inner catheter 401 can vary to accommodate variations in human anatomy. In some embodiments, the length of inner catheter 401 can be approximately several centimeters longer than the outer sheath in the same package. For example, the length of inner catheter 401 can be about 8 to 10 centimeters longer than the outer sheath.

In some embodiments, the distal tip 403 of the inner catheter 401 can be echolucent so that position of the inner catheter 401 can be confirmed by ultrasound. In some embodiments, demarcations 409 with 1 centimeter (or any other unit of measure) gradations are present in order to measure the depth of insertion. The first gradation on the distal side can be at a distance that signifies the length of the outer sheath 301. For example, if the outer sheath 301 is 15 centimeters long, the first gradation on the inner catheter 401 can be 15 centimeters from its distal tip 403. In this way, when the inner catheter 401 is introduced inside the outer sheath 301 to the depth of the first gradation, the distal tip 403 of the inner catheter 401 can be flush with the distal tip 303 of the outer sheath 301. When the inner catheter 401 is introduced further into the outer sheath 301, successive gradations on the inner catheter 401 can indicate the depth that the distal tip 403 has reached beyond the outer sheath. The proximal end of the inner catheter 401 can be fitted with threaded locking mechanisms 405 and 407, so that the inner catheter 401 can be secured distally to an outer sheath and proximally to a standard syringe. Locking mechanisms 405, 407 can be, for example, Luer locks.

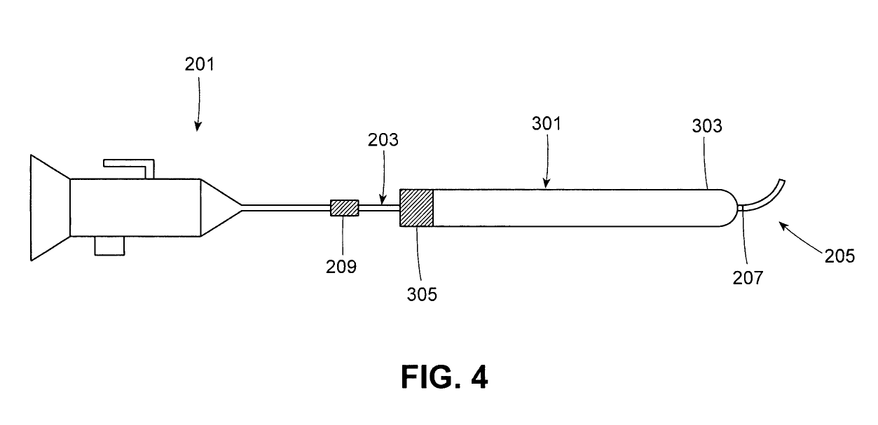

FIGS. 4-6 illustrate an example deliverable delivery procedure using the microfiberoptic catheter to deliver embryo into a uterus. More specifically, FIG. 4 illustrates the first stage of the procedure. As shown, microfiberoptic 203 of the microfiberoptic endoscope 201 can be fitted inside the outer sheath 301. The distal tip 303 of the outer sheath 301 can be aligned with the demarcater 207 on the microfiberoptic endoscope 201, leaving the distal tip 205 of the microfiberoptic endoscope 201 outside the outer sheath 301. The threaded connector 209 of the microfiberoptic endoscope 201 can be used to lock with the locking mechanism 305 of the outer sheath 301, so that the microfiberoptic 203 is secured inside the outer sheath 301. Before insertion of the microfiberoptic endoscope 201 and the outer sheath 301, the patient can be positioned and prepped per usual procedure for an embryo transfer, and the devices can be sterilized. Prepping a patient can include, for example, comfortably positioning the patient in dorsal lithotomy position, placing a speculum in the vagina of the patient, and aseptically cleansing the cervix and vagina of the patient.

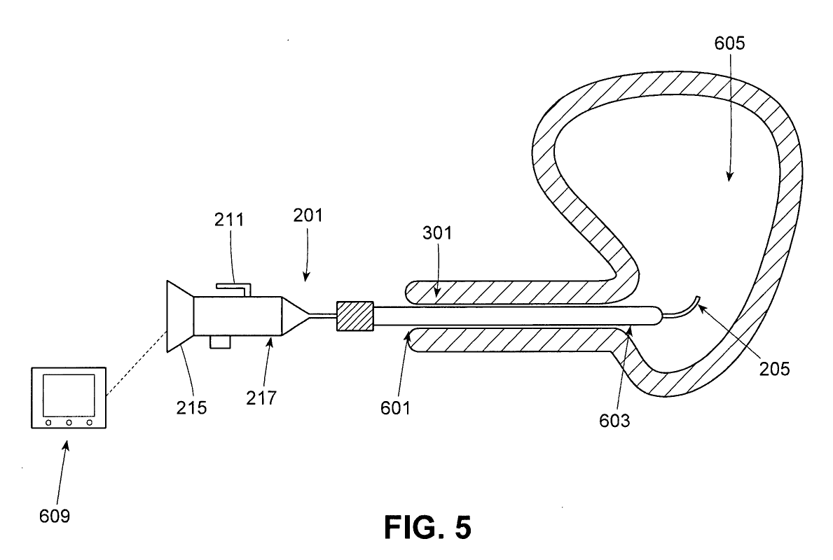

FIG. 5 illustrates the second stage of the procedure, wherein the outer sheath 301 and the distal portion of the microfiberoptic endoscope 201 have been inserted through the external opening 601 of the cervical canal. As shown, the distal tip 205 of the endoscope 201 has reached beyond the internal opening 603 of the cervical canal so that it is inside the endometrial cavity 605. This can been accomplished by using the steering lever 211 on the endoscope 201 to steer and negotiate a passage through the cervical canal and the endometrial cavity. In some embodiments, a video camera and a video monitor 609 can be attached to the endoscope 201 through the coupler 215 to display images provided by endoscope 201, allowing direct visualization during the insertion. Alternatively, a person performing the procedure can directly look through the eyepiece 217 of the endoscope 201.

The microfiberoptic endoscope 201 can provide direct visualization of a steeply flexed junction between the cervix and the body of the uterus. The flexibility of the microfiberoptic and the outer sheath 301, as well as the steering of the distal tip 205, may allow the endoscope 201 and the outer sheath 301 to negotiate obstacles during the insertion through the cervical canal and the endometrial cavity. In some embodiments, after the endoscope 201 reaches beyond the internal opening 603 of the cervical canal, the outer sheath 301 can be inserted over the endoscope 201. Alternatively, the endoscope 201 can be inserted together with the outer sheath 301. According to some embodiments, the depth of insertion of the outer sheath 301 can be determined by gradations on the surface of the outer sheath 301. In some embodiments, the distal tip 303 of the outer sheath 301 can be echolucent and the position of the distal tip 303 can be determined by ultrasound. After the outer sheath 301 reaches a desired depth, the endoscope 201 can be removed, with the outer sheath 301 left in place.

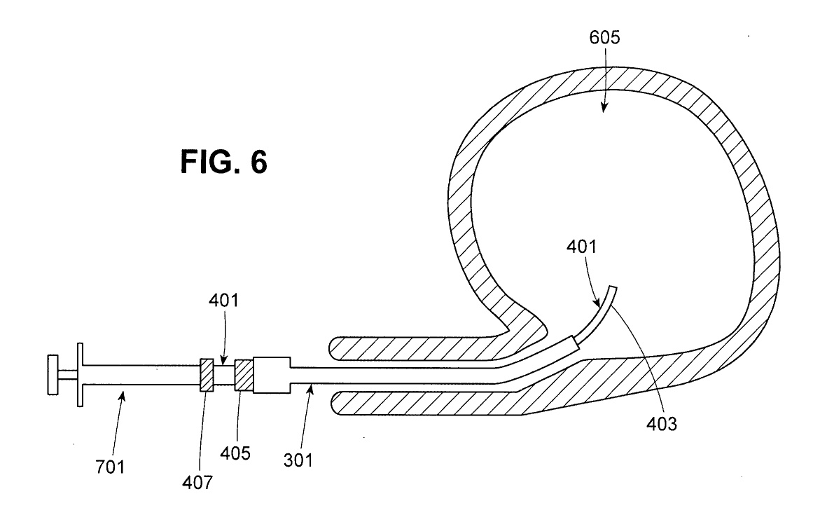

FIG. 6 illustrates the third stage in the procedure, wherein the microfiberoptic endoscope has been removed, with the outer sheath 301 left in place. The inner catheter 401 can be inserted through the outer sheath 301 and introduced to the appropriate depth. In some embodiments, the depth of insertion of the inner catheter 401 can be determined by gradations on the inner catheter 401 and the outer sheath 301. In some embodiments, the distal tip 403 of the inner catheter 401 can be echolucent and the position of the distal tip 403 can therefore be determined by ultrasound. The inner catheter 401 can be locked to the outer sheath 301 through the locking mechanism 405. A syringe 701 can be locked with the inner catheter 401 through the locking mechanism 407. The inner catheter 401 can be loaded with embryos in a volume of fluid determined by an embryologist, and the syringe 701 can be used to inject the embryos and the fluid into the endometrial cavity, or to implant the embryos onto the endometrial lining. After the injection, both the outer sheath 301 and inner catheter 401 can be removed together.

As FIGS. 4-6 illustrate, in some embodiments, the microfiberoptic endoscope 201 and the inner catheter 401 are not inserted into the endometrial cavity 605 at the same time. Instead, each piece can fit inside the outer sheath 301 and can be inserted one at a time. This allows the outer sheath 301 to be thin, because the inner diameter of the outer sheath 301 only needs to be slightly larger than the larger of the diameter of the microfiberoptic 203 of the microfiberoptic endoscope 201 and the outer diameter of the inner catheter 401.

However, in other embodiments, microfiberoptic endoscope 201 and inner catheter 401 can simultaneously fit inside the outer sheath 301. The combination of microfiberoptic endoscope, inner catheter 401, and outer sheath 301 can then be simultaneously inserted into the human body so that delivery of the deliverables can be monitored after insertion into the human body.

It will be readily apparent to one of ordinary skill in the art that systems and methods according to some embodiments can be used to deliver any suitable deliverables, such as an egg, sperm, gamete, fertilized egg, zygote, embryo, and the like, to any suitable and/or desired location within the human body, such as the uterus, the fallopian tube, and the like.

Other embodiments, extensions, and modifications of the ideas presented above are comprehended and within the reach of one skilled in the field upon reviewing the present disclosure. Accordingly, the scope of the present invention in its various aspects is not to be limited by the examples, applications, and embodiments presented above. The individual aspects of the present invention, and the entirety of the invention are to be regarded so as to allow for modifications and future developments within the scope of the present disclosure. Various features of the invention can be used in any suitable combination. The present invention is limited only by the claims that follow.

What is claimed is:

1. An apparatus for delivering one or more deliverables into a location in a body, the apparatus comprising:
a sheath;
an endoscope having a distal portion and a proximal portion and including a microfiberoptic disposed at the distal portion, said endoscope being capable of being inserted at least partially within the sheath, and capable of guiding insertion of the sheath into a configuration in the body; and
a catheter capable of being inserted into the sheath, and capable of delivering one or more deliverables to the location in the body based upon the configuration of the sheath;
wherein the inner diameter of the outer sheath is less than the sum of the outer diameter of the microfiberoptic and the outer diameter of the catheter.

2. The apparatus of claim 1, wherein the endoscope comprises a steering lever operably configured to steer a distal tip of the microfiberoptic.

3. The apparatus of claim 1, further comprising a video monitoring device optically coupled to the endoscope.

4. The apparatus of claim 1, wherein a distal tip of the sheath is beveled.

5. The apparatus of claim 1, wherein at least one of a distal tip of the endoscope, a distal tip of the catheter, and a distal tip of the sheath is capable of being imaged using an imaging technique.

6. The apparatus of claim 5, wherein the imaging technique is ultrasound.

7. The apparatus of claim 1, wherein the catheter comprises at least one demarcator indicating a depth of insertion of the catheter when the catheter is inserted into the sheath.

8. The apparatus of claim 1, wherein at least one of the sheath and the endoscope comprises a connector capable of securing the endoscope to the sheath.

9. The apparatus of claim 8, wherein the sheath comprises a first threaded connector and the endoscope comprises a second threaded connector, wherein the first threaded connector and the second threaded connector are capable of securing the endoscope to the sheath.

10. The apparatus of claim 9, wherein the first threaded connector and the second threaded connector comprise Luer locks.

11. The apparatus of claim 9, wherein a location of the second threaded connector on the endoscope is capable of being adjusted.

12. The apparatus of claim 1, wherein at least one of the sheath and the catheter comprises a connector capable of securing the catheter to the sheath.

13. The apparatus of claim 12, wherein the sheath comprises a first threaded connector and the catheter comprises a third threaded connector, wherein the first threaded connector and the third threaded connector are capable of securing the catheter to the sheath.

14. The apparatus of claim 13, wherein the first threaded connector and the third threaded connector comprise Luer locks.

15. The apparatus of claim 13, wherein a location of the third threaded connector on the catheter is capable of being adjusted.

16. The apparatus of claim 1, wherein the deliverables are selected from at least one of an embryo, a gamete, a fertilized egg, and a zygote.

17. A method for delivering one or more deliverables into a location in a body, the method comprising:
inserting an endoscope inside a sheath;
inserting the endoscope and the sheath into the body;
removing the endoscope from the sheath while leaving the sheath in the body;
inserting a catheter capable of carrying one or more deliverables into the sheath; and
delivering the deliverables to the location in the body using the catheter.

18. The method of claim 17, wherein said inserting the endoscope and the sheath comprises inserting the endoscope to at least one of an endometrial cavity and a fallopian tube of the body.

19. The method of claim 17, further comprising determining, by ultrasound, a position of at least one of the sheath and the catheter inside the body.

20. The method of claim 17, further comprising connecting one of the endoscope and the catheter to the sheath.

21. An apparatus for delivering one or more deliverables into a location in a body, the apparatus comprising:
means for inserting an endoscope inside a sheath;
means for inserting the endoscope and the sheath into the body;
means for removing the endoscope from the sheath while leaving the sheath in the body;
means for inserting a catheter capable of carrying one or more deliverables into the sheath; and
means for delivering the deliverables to the location in the body using the catheter.
